(19) United States
(12) Patent Application Publication
(10) Pub. No.: US 2023/0147592 A1
(43) Pub. Date: May 11, 2023

(54) ALTERATIONS IN THE MOLECULAR COMPOSITION OF URINE FROM COVID19 PATIENTS, DETECTED USING RAMAN SPECTROSCOPIC AND COMPUTATIONAL ANALYSIS

(71) Applicant: Virginia Tech Intellectual Properties, Inc., Blacksburg, VA (US)

(72) Inventors: John L. Robertson, Floyd, VA (US); Ryan Senger, North Chesterfield, VA (US)

(21) Appl. No.: 17/982,019

(22) Filed: Nov. 7, 2022

Related U.S. Application Data
(60) Provisional application No. 63/276,872, filed on Nov. 8, 2021.

(51) Int. Cl. G01N 33/493; G01N 21/65; A61B 5/00

(52) U.S. Cl. CPC G01N 33/493; G01N 21/65; A61B 5/7264

(57) ABSTRACT

The present invention comprises methods of detecting and classifying COVID-19 disease using Raman spectra obtained from subject urine samples. Raman spectra from subject urine samples are analyzed using models prepared from reference Raman samples obtained from urine samples of individuals with and without COVID-19. The spectral fingerprints of urine from subjects with and without COVID-19 allow for identification of disease-associated changes in urine molecular composition.

### CROSS-REFERENCE TO RELATED APPLICATIONS

This application relies on the disclosure of and claims priority to and the benefit of the filing date of U.S. Provisional Application No. 63/276,872 filed Nov. 8, 2021, which is hereby incorporated by reference herein in its entirety.

Additionally, the present application is related to U.S. patent application Ser. No. 15/305,940, filed Oct. 21, 2016, Ser. No. 17/146,301, filed Jan. 11, 2021, and Ser. No. 17/188,737, filed Mar. 1, 2021, each of which is incorporated by reference herein in its entirety.

### BACKGROUND OF THE INVENTION

### Field of the Invention

The present invention relates to the field of clinical care of COVID-19 patients. More particularly, the present invention relates to a system and method for diagnosing COVID-19 and/or monitoring disease state with Raman spectroscopy measurements.

### Description of Related Art

Infection with SARS-CoV-2 and development of COVID-19 disease is associated with a deleterious effect in renal function and structure. This would be expected to potentially alter the molecular composition of urine. Since COVID-19 evolved in 2019, there have been numerous reports of acute kidney injury (AKI) associated with this infection (Batlle, D. et al. COVID-19 and ACE2 in Cardiovascular, Lung, and Kidney Working Group: Acute kidney injury in COVID-19: Emerging evidence of a distinct pathophysiology. J. Am. Soc. Nephrol. 2020; 31: 1380-1383; Chen, Y. T. et al. Mortality rate of acute kidney injury in SARS, MERS, and COVID-19 infection: A systematic review and meta-analysis. Crit. Care 2020; 24: 439; Cheng, Y. et al. Kidney disease is associated with in-hospital death of patients with COVID-19. Kidney Int. 2020; 97: 829-838; Ng, J. H. et al. Pathophysiology and pathology of acute kidney injury in patients with COVID-19. Adv. Chronic Kidney Dis. 2020; 27: 365-376; Pei, G. et al. Renal involvement and early prognosis in patients with COVID-19 pneumonia. J. Am. Soc. Nephrol. 2020; 31: 1157-1165; Richardson, S. et al. Presenting characteristics, comorbidities, and outcomes among 5700 patients hospitalized with COVID-19 in the New York City area. JAMA 2020; 323: 2052-2059; Sharma, P. et al. COVID-19-associated kidney injury: A case series of kidney biopsy findings. J. Am. Soc. Nephrol. 2020; 31: 1948-1958; Su, H. et al. Renal histopathological analysis of 26 postmortem findings of patients with COVID-19 in China. Kidney Int. 2020; 98: 219-227). The incidence of AKI in COVID-19 patients has been estimated to range from about 27-50+% (Hirsch, J. S. et al. Acute kidney injury in patients hospitalized with COVID-19. Kidney Int. 2020; 98: 209-218; Mohamed, M. M. et al. Acute kidney injury associated with coronavirus disease 2019 in urban New Orleans. Kidney 360 2020; 1: 614-622). Early in the pandemic, several groups noted a correlation of disease severity, hospitalization, and intensive care admissions with increased risk for developing AKI (Fisher, M. et al. AKI in hospitalized patients with and without COVID-19: A comparison study. J. Am. Soc. Nephrol. 2020; 31: 2145-2157; Moledina, D. G. et al. The association of COVID-19 with acute kidney injury independent of severity of illness: A multicenter cohort study. Am. J. Kidney Dis. 2021; 77: 490-499.e1). This was not surprising. The contribution of cardiopulmonary dysfunction, renal hypoperfusion, and/or multidrug therapy leading to the development AKI has been well-known for decades prior to COVID-19 (Murugan, R. and Kellum, J. A. Acute kidney injury: what's the prognosis? Nat. Rev. Nephrol. 2011; 7(4): 209-217). However, the role of renal viral infection in the development of AKI was uncertain.

A recent review by Hassler and coworkers considered evidence both for and against direct SARS-CoV-2 infection of the kidney (Hassler, L. et al. Evidence for and against direct kidney infection by SARS-CoV-2 in patients with COVID-19. CJASN 2021; 16). Rightly so, Hassler, et. al., and authors they cited, felt that viral infection might help explain the disproportionately high incidence of AKI and collapsing glomerulopathy seen in patients with COVID-19. Combining data from several studies, Hassler, et. al., presented at least putative evidence of viral infection in 102/235 kidneys (43%) from autopsied patients. A variety of techniques for viral/viral RNA detection were used in the cited studies, including immunohistochemistry, real-time polymerase chain reaction (RT-PCR), in situ hybridization, immunofluorescent microscopy, and electron microscopy. No study they referenced used two or more methods for cross-checking and validating renal viral infection, a deficit in study design considered to affect interpretation in the results. Hassler et al. posited that renal biopsies (not autopsy-derived samples) and development of urine-based screening tests would be keys to understanding the effects of COVID-19 on renal function and structure and these would be needed to improve detection and management of disease (Hassler, 2021).

A need remains for a urine-based screening test for diagnosing and understanding the renal effects of COVID-19.

### SUMMARY OF THE INVENTION

In embodiments, the present invention comprises a method to detect COVID-19 disease using urine specimens. The technology is based on Raman spectroscopy and computational analysis. It does not detect SARS-CoV-2 virus or viral components, but rather a urine ‘molecular fingerprint’, representing systemic metabolic, inflammatory, and immunologic reactions to infection.

Embodiments of the invention include Aspect 1, which is a method for determining COVID-19 status of a subject, comprising: obtaining a test Raman spectrum from a urine sample from a test subject; obtaining at least one positive reference Raman spectrum from a urine sample from a COVID-19 positive subject and at least one negative reference Raman spectrum from a urine sample from a COVID-19 negative subject; identifying at least one significant Raman shift; analyzing the test Raman spectrum, positive Raman spectrum, and negative Raman spectrum by performing principal component analysis (PCA) and/or discriminant analysis of principal components (DAPC) using the at least one significant Raman shift; and classifying the test subject as COVID-19 positive or COVID-19 negative based on the analyzing.

Aspect 2 is the method of Aspect 1, further comprising processing the test Raman spectrum, positive Raman spectrum, and/or negative Raman spectrum to obtain a processed test Raman spectrum, a processed positive Raman spectrum, and/or a processed negative Raman spectrum.

Aspect 3 is the method of Aspects 1 or 2, wherein the processing comprises a baseline correction.

Aspect 4 is the method of any of Aspects 1-3, wherein the baseline correction is performed using ISREA.

Aspect 5 is the method of any of Aspects 1-4, wherein the baseline correction comprises selecting one or more ISREA nodes selected from 400, 439, 446, 605, 950, 1045, 1100, 1163, 1247, 1443, 1500, 1739, 1768, 1775, and 1800 cm⁻¹.

Aspect 6 is the method of any of Aspects 1-5, wherein the processing comprises truncating the test Raman spectrum, positive Raman spectrum, and/or negative Raman spectrum.

Aspect 7 is the method of any of Aspects 1-6, wherein the truncating is performed between about 600-1,800 cm⁻¹.

Aspect 8 is the method of any of Aspects 1-7, wherein the significant Raman shift is defined as a shift having above 0.2% of a total contribution to a Raman spectrum.

Aspect 9 is the method of any of Aspects 1-8, wherein the at least one significant Raman shift is selected from 425 cm⁻¹, 445 cm⁻¹, 485 cm⁻¹, 518 cm⁻¹, 614 cm⁻¹, 621 cm⁻¹, 627 cm⁻¹, 682 cm⁻¹, 688 cm⁻¹, 702 cm⁻¹, 719 cm⁻¹, 776 cm⁻¹, 782 cm⁻¹, 810 cm⁻¹, 817 cm⁻¹, 830 cm⁻¹, 847 cm⁻¹, 860 cm⁻¹, 880 cm⁻¹, 893 cm⁻¹, 900 cm⁻¹, 906 cm⁻¹, 913 cm⁻¹, 955 cm⁻¹, 980 cm⁻¹, 992 cm⁻¹, 1002 cm⁻¹, 1006 cm⁻¹, 1008 cm⁻¹, 1013 cm⁻¹, 1030 cm⁻¹, 1049 cm⁻¹, 1058 cm⁻¹, 1073 cm⁻¹, 1077 cm⁻¹, 1080 cm⁻¹, 1104 cm⁻¹, 1107 cm⁻¹, 1126 cm⁻¹, 1185 cm⁻¹, 1240 cm⁻¹, 1327 cm⁻¹, 1396 cm⁻¹, 1491 cm⁻¹, 1607 cm⁻¹, 1630 cm⁻¹, and 1641 cm⁻¹.

Aspect 10 is the method of any of Aspects 1-9, wherein the analyzing further comprises identifying statistically significant differences between the test Raman spectrum and the positive and/or negative reference spectra.

Aspect 11 is the method of any of Aspects 1-10, wherein the identifying statistically significant differences comprises performing one or more of total canonical distance (TCD), total principal component distance (TPD), or total spectra distance (TSD).

Aspect 12 is a method of identifying a condition of a subject, comprising: obtaining Raman spectra from a urine sample from a subject; comparing the Raman spectra of the urine sample to a selected model; wherein the selected model is constructed from various Raman spectra of urine from individuals having and not having COVID-19, and by: (a) applying baseline correction to a range of wavenumbers of the various Raman spectra to obtain baseline corrected Raman spectra; (b) performing normalization of the baseline corrected Raman spectra to obtain normalized Raman spectra; (c) performing principal component analysis (PCA) of the normalized Raman spectra to identify principal components (PCs) of the urine from the individuals having and not having COVID-19; (d) performing one or more analysis selected from discriminant analysis of principal components (DAPC), Partial Least Squares (PLS), machine learning, and/or neural networks (NN), to obtain one or more chemometric models based on one or more of the PCs and for the DAPC analysis, comprising canonicals equal in number to the PCs; (e) for the DAPC analysis, determining a fractional contribution of each wavenumber to each canonical of one or more of the chemometric models to determine which wavenumbers give rise to separations seen in a plot of two or more of the canonicals; and (f) identifying statistically significant spectral differences between the urine from the individuals having the specified condition and the urine from individuals not having COVID-19 by performing total principal component distance (TPD) and/or total spectral distance (TSD); wherein the comparing of the Raman spectra of the urine sample to the selected model comprises identifying whether the urine sample is classified according to the selected model as being urine either from a subject who has or does not have COVID-19.

Aspect 13 is the method of Aspect 12, wherein the baseline correction is performed using ISREA.

Aspect 14 is the method of Aspects 12 or 13, wherein the baseline correction comprises choosing ISREA nodes, wherein the ISREA nodes are selected from 400, 950, 110, 1500, and 1800 cm⁻¹.

Aspect 15 is the method of any of Aspects 12-14, wherein multiple chemometric models are obtained with different numbers of principal components.

Aspect 16 is the method of any of Aspects 12-15, further comprising testing one or more of the chemometric models using a leave-one-out cross-validation technique to select one of the chemometric models as the selected model.

Aspect 17 is the method of any of Aspects 12-16, wherein the selected model is one where the DAPC model is based on up to about 20 PCs.

Aspect 18 is the method of any of Aspects 12-17, wherein the selected model is one where the DAPC model is based on up to about 5 PCs.

Aspect 19 is the method of any of Aspects 12-18, wherein the selected model is constructed from various Raman spectra from individuals classified as having mild COVID-19 symptoms lasting less than 30 days, moderate COVID-19 symptoms lasting less than 30 days, severe COVID-19 symptoms lasting less than 30 days, or COVID-19 symptoms lasting 30 days or more.

Aspect 20 is the method of any of Aspects 12-19, wherein when the urine sample is classified according to the selected model as being urine from a subject who has COVID-19, further classifying the urine sample as being from a subject having mild COVID-19 symptoms lasting less than 30 days, moderate COVID-19 symptoms lasting less than 30 days, severe COVID-19 symptoms lasting less than 30 days, or COVID-19 symptoms lasting 30 days or more.

### BRIEF DESCRIPTION OF THE DRAWINGS

The accompanying drawings illustrate certain aspects of implementations of the present disclosure, and should not be construed as limiting. Together with the written description the drawings serve to explain certain principles of the disclosure.

FIG. 1A is a graph showing Raman metabolic fingerprints of human urine.

FIG. 1B is a graph showing the discriminant analysis of principal components (DAPC) for the Raman metabolic fingerprints of FIG. 1A.

FIG. 1C is a graph showing the partial least squares regression (PLSR) for several metabolomic biomarkers.

FIG. 2 is a graph showing ISREA baselined and vector normalized spectra for each of the classes specified in Table 1.

FIGS. 3A-B are DAPC models demonstrating cluster separation of COVID-19 Raman urine spectra from those of other groups.

FIG. 3C is a graph showing the performance of several DAPC models.

FIGS. 4A-B are DAPC models demonstrating cluster separation of COVID-19 Raman urine spectra by clinical severity (FIG. 4A) and duration of symptoms (FIG. 4B).

### DETAILED DESCRIPTION OF VARIOUS EMBODIMENTS OF THE INVENTION

Normal human urine contains over 2,000 separate chemical entities, reflective of systemic physiology/metabolism and the processes of renal function (Bouatra, S. et al. The human urine metabolome. PLoS One. 2013; September 4; 8(9): e73076). In the past decade, mass spectrometry, liquid/gas chromatography, nuclear magnetic resonance, and kinetic nephelometry methods have been used to detect analytes (i.e., biomarkers) associated with normal metabolism or disease (Emwas, A. H. et al. Standardizing the experimental conditions for using urine in NMR-based metabolomic studies with a particular focus on diagnostic studies: a review. Metabolomics 2015; 11(4): 872-894). Metabolomics has been used to identify kidney disorders (including renal cell carcinoma), coronary artery disease, diabetes, Alzheimer's disease and cognitive impairment, neurodegenerative disease, and colorectal cancer (Castelli, F. A. et al. Metabolomics for personalized medicine: the input of analytical chemistry from biomarker discovery to point-of-care tests. Anal. Bioanal. Chem. 2021; 25: 1-31; Chen, Z. and Kim, J. Urinary proteomics and metabolomics studies to monitor bladder health and urological diseases. BMC Urol. 2016; 16 (March 22): 11; Cheng, S. et al. Potential impact and study considerations of metabolomics in cardiovascular health and disease: A scientific statement from the American Heart Association. Circ. Cardiobasc. Genet. 2017; 10(2): e000032; Dator, R. et al. Metabolomics profiles of smokers from two ethnic groups with differing lung cancer risk. Chem. Res. Toxicol. 2020; 33(8):2087-2098; Gowda, G. A. et al. Metabolomics-based methods for early disease diagnostics. Expert Rev. Mol. Diagn. 2008; 8(5):617-633; Turi, K. N. et al. A review of metabolomics approaches and their application in identifying casual pathways of childhood asthma. J. Allergy Clin. Immunol. 2018; 141(4):1191-1201). While metabolomics is often used to search for circulating/plasma disease biomarkers, it is now used to study how the presence of disease alters the urine metabolite profile (“fingerprint”).

Mass spectrometry-based urine biomarker and “-omics” technologies are used rarely by caregivers in patient care settings. This is due to expense, the daunting requirement for advanced technology, expertise required for interpretation of results, and the lack of assay validation requiring large datasets of normal and abnormal specimens. In fact, the complexity of both acute and chronic genitourinary tract pathologies makes large dataset sampling and validation with technology-intensive methods (like mass spectrometry and high-performance liquid chromatography) unlikely and cost-prohibitive.

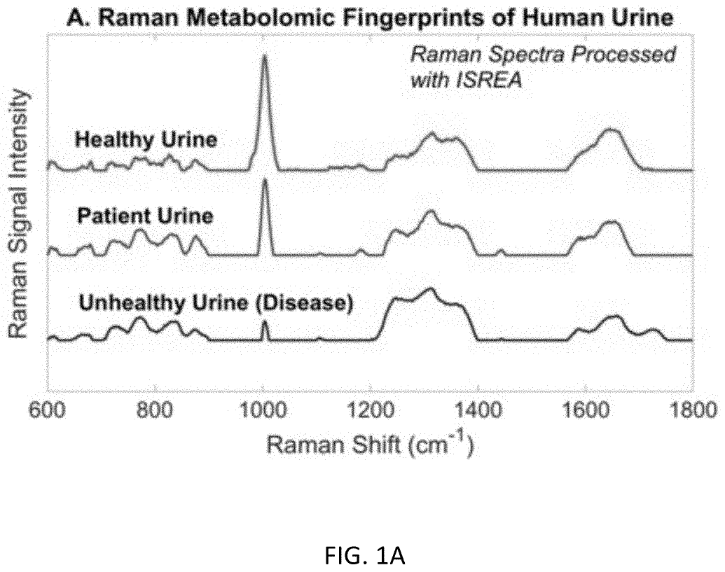

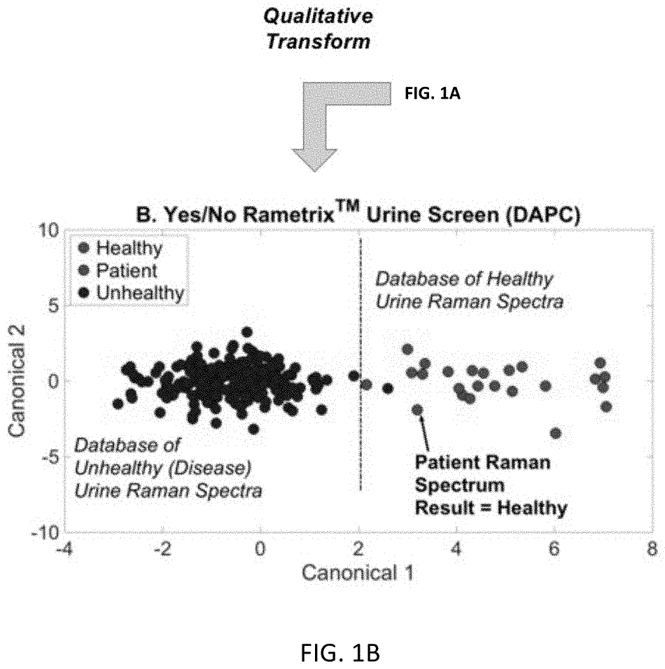

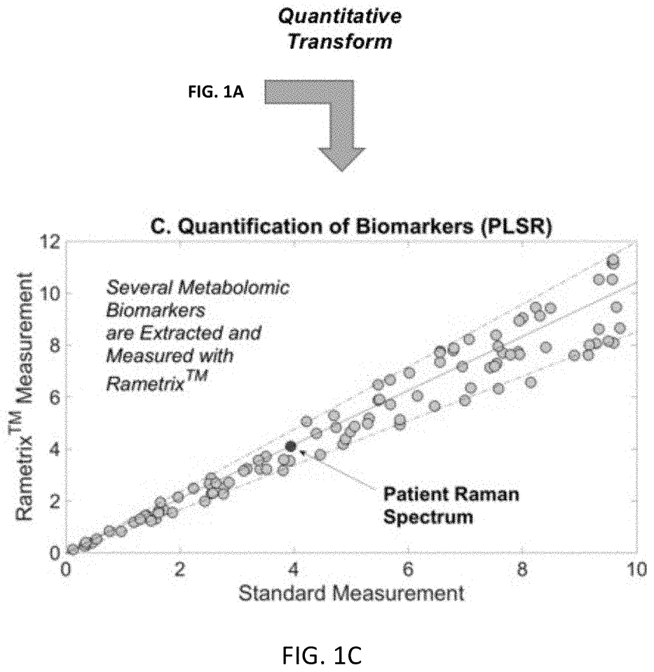

As an alternative approach to mass spectroscopy-based urine metabolomics, the present inventors have developed an approach to molecular urinalysis, using a combination of Raman spectroscopic, computational, and physicochemical analytical methods (Senger, R. et al. Spectral characteristics of urine specimens from healthy human volunteers analyzed using Raman Chemometric Urinalysis (Rametrix). PLoS One. 2019; 14(9): e0222115; Senger, R. et al. Spectral characteristics of urine from patients with end-stage kidney disease, analyzed using Raman Chemometric Urinalysis (Rametrix). PLoS One. 2020; 15(1): e0227281; Carswell, W. et al. Raman Spectroscopy as a non-cytological detection and quantification urinalysis method for microhematuria in human urine. Appl. Spectro. 2022. 76(3): 1-11; Huttanus, H. et al. Raman Chemometric Urinalysis (Rametrix) as a screen for bladder cancer. PLoS One. 2020; 15(8): e0237070; Senger, R. et al. Disease-associated multimolecular signature in the urine of patients with Lyme disease, detected using Raman spectroscopy and chemometrics (Rametrix). Appl. Spectro. 2022 76; 284-299; Fisher, A. K. et al. The Rametrix LITE Toolbox v1.0 for MATLAB®. J. Raman Spectrosc. 2018; 49: 885-896; Senger, R. and Robertson, J. The Rametrix PRO Toolbox V1.0 for MATLAB®. Peer J. 2020; 8: e8179). Raman spectroscopy is a powerful technology that can be used for analysis of the chemical composition of solids and liquids, including biological specimens (Celia-May, D. et al. Theoretical principles of Raman spectroscopy. Phys. Sci. Rev.: 2019; 19:4; Movasaghi, Z. et al. Raman spectroscopy of biological tissues. Appl. Spectrosc. Rev. 2007; 42: 493-541; Senger, R. S. and Scherr, D. Resolving complex phenotypes with Raman spectroscopy and chemometrics. Curr. Opin. Biotechnol. 2020; 66:277-282; Shen, Y. et al. Raman imaging of small biomolecules. Annu. Rev. Biophys. 2019; 48:347-369). Irradiation of molecular mixtures (like urine), with wavelength-specific laser energy, produces weak vibrational energy (Raman scatter radiation) from deformation/relaxation of the many chemical bonds in hundreds of distinct molecules in specimens. Different molecular constituents are represented by Raman ‘bands’ (i.e., signal intensity peaks) and these bands/peaks are indicative of chemical bond vibrations (Gardiner, D. Practical Raman Spectroscopy. NY, Springer-Verlag; 1989). These vibrations may be present in several molecules with similar chemical bonds in a sample, meaning it can be difficult to assign individual Raman bands to specific molecules, unless they are present in abundance. This is the case for urea in urine, for example, where the C—N bond stretch at 1,002 cm′ is dominant and can be associated with urea concentration. The bands/peaks of creatinine, heme, amino acids, albumin, collagen, and phospholipids in urine have also been identified. A few of these and other broad molecular assignments are shown in Raman spectra of urine from healthy volunteers (Senger, 2019), CKD 4-5 patients (Senger, PLoS One 2020), and Surine™ urinalysis analytical control solution (Dyna-Tek Industries, Lenexa, Kans.) in FIGS. 1A-C.

Because it is difficult to relate individual Raman bands to specific molecules, a chemometric approach is required to analyze Raman spectra of highly complex heterogenous samples (Fisher, 2018; Athamneh, A. I. M. and Senger, R. S. Peptide-guided Surface-Enhanced Raman Scattering probes for localized cell composition analysis. Appl. Env. Microbiol. 2012; 78: 7805-7808; Athamneh, A. I. M. et al. Phenotypic profiling of antibiotic response signatures in Escherichia coli using Raman spectroscopy. Antimicrob. Agents Chemother. 2014; 58: 1302-1314; Lussier, F. et al. Deep learning and artificial intelligence methods for Raman and surface-enhanced Raman scattering. Trends Anal. Chem. 2020; 124:115796; Zu, T. N. K. et al. Near-real-time analysis of the phenotypic responses to Escherichia coli to 1-butanol exposure using Raman spectroscopy. J. Baceriol. 2014; 196: 3983-3991; Zu, T. N. K. et al. Assessment of ex vivo perfused liver health by Raman Spectroscopy. J. Raman. Spectrosc. 2015; 46: 551-558). The chemometric approach is unlike chromatographic and mass spectrometry approaches that resolve single molecules. The chemometric approach treats an entire Raman spectrum as a ‘fingerprint’ and then associates it with a condition (i.e., ‘healthy’, ‘chronic kidney disease,’ ‘COVID-19 infection’, etc.) using statistical models and artificial intelligence. Building an accurate model to predict the condition of an unknown sample requires a large dataset of pre-analyzed Raman spectra. This can be seen, for example, in FIGS. 1A-C. Here, representative urine spectra are shown for healthy volunteers and patients with diagnosed disease (FIG. 1A). Chemometric models determine whether an “unknown” patient sample more closely resembles the healthy urine spectrum or one of a diseased state, without knowing the identities of all molecules in each sample (FIGS. 1B-C).

Rametrix® computations are performed using the Rametrix® Toolbox for MATLAB, which is available to academic researchers through GitHub (Fisher, 2018; Senger, Peer J. 2020). The Rametrix® Toolbox offers two approaches to data analysis (also shown in FIGS. 1A-C), (i) qualitative classification, and (ii) quantitative analysis. For classification (e.g., “yes/no” to the presence of disease) the Rametrix® Toolbox offers principal component analysis (PCA) followed by discriminant analysis of principal components (DAPC) (Fisher, 2018; Senger, Peer J. 2020; Senger, Curr. Opin. Biotechnol. 2020). Other deep learning and artificial neural network options are available, and other classifier models used with Raman spectra were surveyed recently (Senger, Curr. Opin. Biotechnol. 2020). Partial least-squares regression (PLSR) for quantitative analyses, and methods to identify quantifiable biomarkers from Raman spectra have been produced recently (Carswell, 2022). The Rametrix® Toolbox also includes ISREA (Xu, Y. et al. ISREA: An Efficient Peak-Preserving Baseline Correction Algorithm for Raman Spectra. Appl. Spectro. 2021; 75(1):34-45; Xu, Y. et al. Sparse logistic regression on functional data. Stat. Interface, accepted, 2021), which enables for the “exclusion” of non-diagnostic features in samples/spectra, such as the presence of blood/breakdown products, if such exclusion is warranted and logical (Carswell, 2022; Senger, Curr. Opin. Biotechnol. 2020). Finally, the Rametrix® Toolbox offers a graphical interface, spectral viewing pane, and predictive model cross-validation through leave-one-out analysis (Senger, Peer J. 2020).

Analysis of urine by Rametrix® is inexpensive (uses off-the-shelf Raman spectrometers and costs a few dollars a sample for consumables and analysis), rapid (typically less than 30 minutes to process and interpret a sample), requires no urine sample preparation or chemical manipulation, and is non-invasive (voided samples are analyzed). These characteristics, and ease-of-use, make this Raman spectroscopy-based technology useful in determining if a COVID-19 ‘molecular fingerprint’ is present in the urine of diseased patients. This fingerprint may be useful in disease detection and patient management (Robertson, J. L. et al. (2022) Alterations in the molecular composition of COVID-19 patient urine, detected using Raman spectroscopic/computational analysis, PLOS ONE 17(7): e0270914).

Forty-six (46) patients, with clinical signs of COVID-19 disease, RT-PCR confirmation of nasopharyngeal infection and/or household/congregate and temporal exposure to RT-PCR confirmed patients, were seen by a primary care physician for disease/symptom management. All patients were symptomatic, but ambulatory, at the time of evaluation. The patient population consisted of 32 female and 14 male patients. The age range of female patients was 18-68 years (average age 47.84 years old) and of male patients was 18-62 years (average age 47.85 years old). As would be expected, the clinical presentation of patients was highly variable, as was the duration and severity of clinical symptoms. Thirty out of 46 patients (30/46) were seen for evaluation (and specimen collection) within the first 14 days of clinical disease. Twelve of 46 patients (12/46) had clinical disease present for 30-300 days, and 10 of 46 patients (10/46) had clinical disease present for 60-300 days. Based on physician evaluation and self-assessment, 25/46 patients presented with ‘mild, symptomatic’ disease, while 14/46 patients presented with ‘moderate, symptomatic’ disease. Seven of 46 patients (7/46) were classified ‘severe, symptomatic’ at the time of presentation and specimen collection. Several patients had pursued multiple avenues of diagnosis and variable courses and types of therapies (including antibiotics) prior to evaluation. Clinical pathology evaluation (serum chemistry) was performed on a subset of 20/33 patients. Seven of 20 patients had mildly elevated serum creatinine values (>0.9 mg/dl; range 0.9-1.34 mg/dl) and 6/7 of these patients also had eGFR<90 mL/min/1.73 m² (range 59-84 mL/min/1.73 m²).

Healthy volunteer control samples were collected prior to the surge of COVID-19. A full analysis of the healthy human volunteer urine dataset was published (Senger, 2019). This dataset contains 235 urine specimens collected from 39 females and 9 males, and all were collected prior to December of 2018. All volunteers were healthy (free of infectious or degenerative disease) at the time of collection and had no history or evidence of renal disease. The population ranged in age from 18 to 70 years, and 87.5% were between 19-22 years of age (median of 21 years). A total of 185 urine spectra were selected randomly from this dataset and used as healthy controls.

For this study, 19 additional urine specimens were collected from healthy volunteers who had been fully vaccinated against COVID-19 and who had no history or evidence of either renal disease or COVID-19 disease.

Control samples from patients with end-stage renal disease (ESRD) were collected prior to the surge of COVID-19. The ESRD patient urine dataset has also been published (Senger, PLoS One 2020). It contains 362 urine specimens from 96 patients receiving treatment for ESRD with peritoneal dialysis therapy. The age range was 24 to 90 years, with a mean of 60 and median of 63.5 years. Twenty (20) spectra were selected randomly from this dataset for use as ESRD patient controls.

Control samples from patients with bladder cancer were also collected prior to the surge of COVID-19. These urine spectra from patients with active or remissive BCa (Huttanus, 2020). The dataset contains 56 urine specimens (one per patient) from patients between 31-91 years old (mean and median of 62 years) were collected. From this dataset, 17 specimens were selected from patients with active BCa for use as BCa patient controls.

Voided, mid-stream urine specimens were collected, frozen immediately at −15° C., and stored at −35° C. until analysis. The suitability of this procedure for preserving samples has been demonstrated (Huttanus, 2020).

Urine specimens were analyzed at room temperature in bulk liquid form using 2 mL screw thread flat bottom borosilicate glass vials (Fisher Scientific). A Wasatch Photonics 785 nm dispersive Raman spectrometer (Wasatch Photonics, Morrisville, N.C.) was used with a Rametrix® AutoScanner (DialySensors, Inc., Blacksburg, Va.) to automate sample scanning. The following settings were used: 25° C., 785 nm laser, 30 s excitation time, 30 mW laser power, 0.2 mm laser spot size, 200-2000 cm′ range, and spectral resolution of 8 cm′ (manufacturer default). Ten scans were obtained per vial. ENLIGHTEN™ software (Wasatch Photonics) was used for spectrometer operation, and molecular contributions investigated with a published database (Liu, J. et al. A novel algorithm for Ramen spectrum baseline correction. Appl. Spectrosc. 2015; 69: 834-842). In all cases, Raman intensity and wavenumber calibrations were performed during each operation of the Raman spectrometer using Surine™ urine analytical control (see below) and published chemometric protocols.

Surine™ Urine Negative Control (Dyna-Tek Industries, Lenexa, Kans.) was used as a control.

Previously published computational methods were used (Senger, 2022; Fisher, 2018; Senger, Peer J. 2020; Liu, 2015) with the Rametrix® Toolbox (LITE v1.1 and PRO v1.0) with added capabilities for ISREA baselining (Xu, Appl. Spectro. 2021; Xu, Stat. Interface 2021). Calculations were performed in MATLAB R2018A (Mathworks; Natick, Mass.). Raman spectra were truncated to 600-1800 cm⁻¹, baseline corrected with ISREA, averaged over the 10 scans for each urine specimen, and vector normalized. ISREA was applied using nodes (or ‘knots’) at wavenumbers of 400, 950, 1100, 1500, and 1800 cm⁻¹. In specified cases, the placement of nodes was also allowed to vary, as described previously (Senger, 2022), to exclude specific Raman shift regions of spectra selectively. Spectra were analyzed by principal component analysis (PCA) and discriminant analysis of principal components (DAPC) in the Rametrix® LITE Toolbox, and models were cross-validated with leave-one-out analysis with Rametrix® PRO. These methods have been implemented and described previously (Senger, 2022; Fisher, 2018; Senger, Curr. Opin. Biotechnol. 2020; Athamneh, 2012; Athamneh, 2014; Lussier, 2020; Zu, 2014; Zu, 2015). This procedure allowed calculation of overall prediction accuracy, sensitivity, specificity, positive-predictive value (PPV), and negative-predictive value (NPV) for detecting COVID-19 in human urine. These metrics have also been defined in previous publications, and here, the RT-PCR nasopharyngeal swab test and/or proximate/congregate/temporal exposure to COVID-19 positive patients is treated as the “Gold-Standard” test when comparing to the Rametrix® urine screen.

Statistical comparisons of spectra were performed through the calculation of total spectral distance (TSD), as has been demonstrated (Senger, 2019; Senger, PLoS One 2020; Huttanus, 2020; Senger, 2022; Senger, Peer J. 2020; Senger, Curr. Opin. Biotechnol. 2020). In calculation of TSD, the difference between each urine spectrum and that of Surine™ was calculated at each wavenumber and summed. One-Way Analysis-of-Variance (ANOVA) and pairwise comparisons using Tukey's honestly significant difference (HSD) procedure were used to determine if TSD values of COVID-19 patient urine were different from those of healthy volunteers and those with other diseases.

Table 1 shows the patient data set, including the numbers of specimens, classifications (healthy, ESRD, BCa, Surine, COVID-19 (mild, moderate, severe, or Long COVID)).

TABLE 1
Summary of Patient Dataset.

| Number | | |
|---|---|---|
| of Urine | | |
| Specimens | Description | Classification |
| 185 | Healthy human volunteers (pre-2019) | Healthy |
| 20 | Peritoneal dialysis patients with CKD 4-5 | ESRD |
| 17 | Patients with active bladder cancer | BCa |
| 6 | Surine ™ (lot from 2016) | Surine |
| 5 | Surine ™ (lot from 2021) | Surine |
| 19 | Healthy human COVID-19 vaccinated volunteers (2021) | Healthy |
| 46 | Patients with active COVID-19 | COVID-19 |
| 25 | Patients with ‘mild’ severity COVID-19 symptoms | COVID-19 (mild) |
| 14 | Patients with ‘moderate’ severity COVID-19 symptoms | COVID-19 |
| | | (moderate) |
| 7 | Patients with ‘severe’ COVID-19 symptoms | COVID-19 (severe) |
| 12 | Patients with COVID-19 clinical disease lasting longer | COVID-19 (Long |
| | than 30 days | COVID-19) |

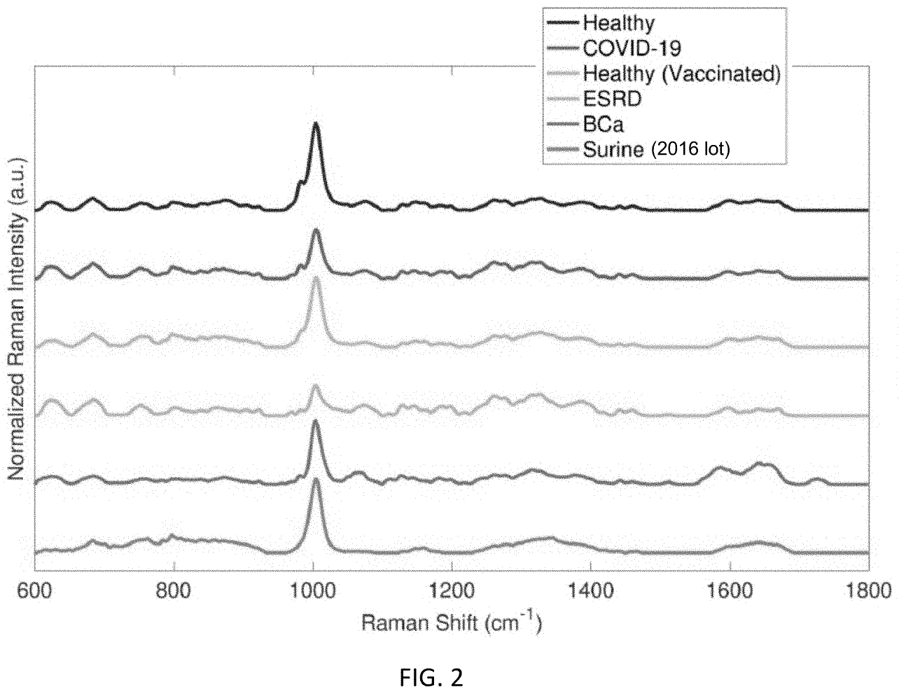

All spectra were truncated between 600-1,800 cm′, baselined using ISREA (Xu, Appl. Spectro. 2021; Xu, Stat. Interface 2021), vector normalized, and averaged for each urine specimen. For the ISREA implementation, nodes were applied at 400, 950, 1100, 1500, and 1800 cm⁻¹. The concept of ISREA node placement and optimization has also been introduced recently (Senger, Curr. Opin. Biotechnol. 2020). Averaged spectra from all classes listed in Table 1 are shown in FIG. 2. The most notable observable difference from the spectra of the COVID-19 and Healthy (pre-2019) classes was the height of the urea representative band (1,002 cm⁻¹). Inspection also revealed other minor differences (e.g., 970 cm⁻¹; 1100-1200 cm⁻¹), prompting additional differences to be investigated by chemometric methods. To date, patients with ESRD have the most visually different urine Raman spectra from the Healthy class (FIG. 2). However, it is noted that the ESRD class is also identified by a reduced urea Raman band intensity.

Total Principal Component Distance (TPD) (Huttanus, 2020; Fisher, 2018; Senger, Peer J. 2020) calculations were performed to determine if urine Raman spectra of the “COVID-19” class were different from all other classes (combined to form a “non-COVID-19” class). In short, TPD uses ISREA baselined and processed spectra. The first five principal components (PCs) of PCA were used. For each sample, the distance (across all five PCs) is calculated between that sample and Surine™ (using a simple distance formula). This provides a distance calculation for every sample in the dataset. Then, ANOVA and pairwise comparisons are used to determine if statistically significant distances exist between classes of spectra. TPD was applied to the COVID-19 class and all other classes grouped as non-COVID-19. Through this method, the COVID-19 class and non-COVID-19 grouped class were found statistically significant (p<0.001). Pairwise comparisons were applied, and the COVID-19 class was found statistically different (p<0.001) against all other groups in Table 1. From experience, this indicates that an effective predictive model may be able to be constructed from PCA followed by DAPC. Of other pairwise comparisons, it was found that the Healthy and Surine™ groups were not statistically different from one another (p=0.79) according to TPD calculations.

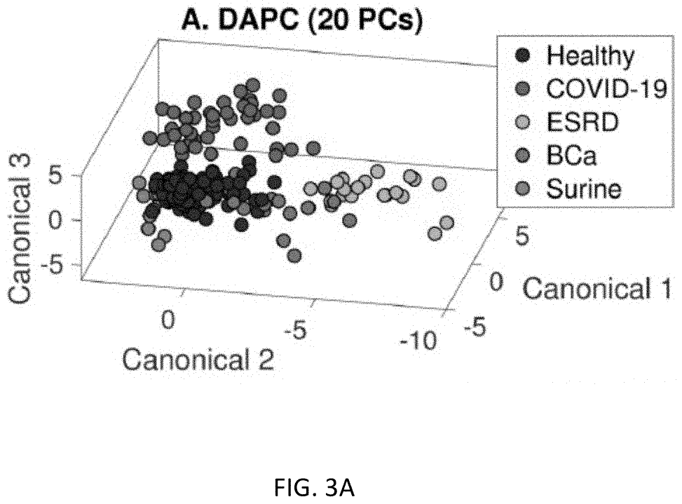

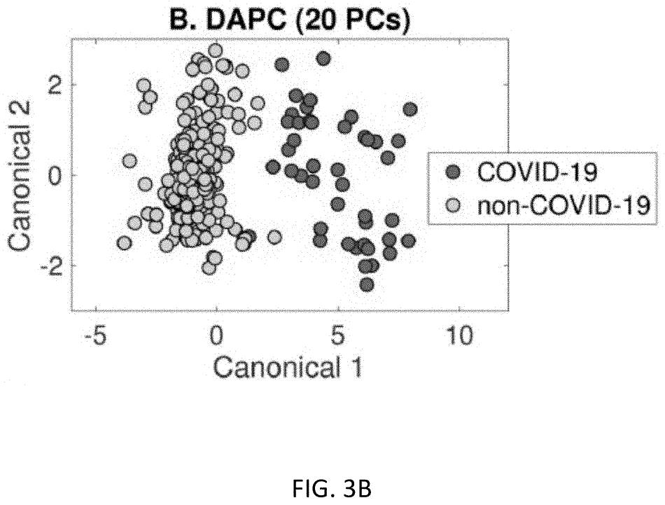

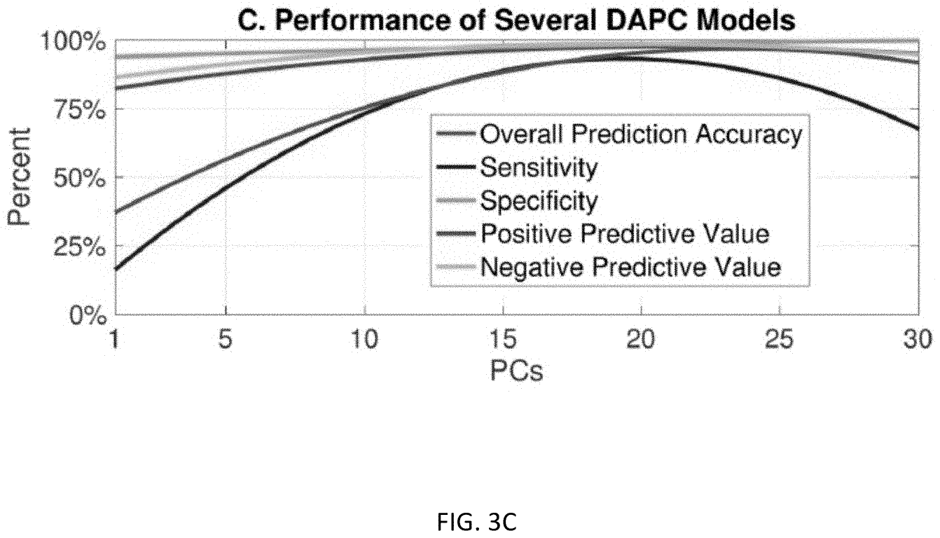

With the COVID-19 class showing statistical significance from all other classes, predictive models were built using PCA and DAPC. The model inputs were truncated, baselined, and normalized urine spectra, and the ISREA nodes of 400, 950, 1100, 1500, and 1800 cm⁻¹ were used in this initial model-building. Spectra were processed further by PCA to produce principal components (PCs). A specified number of PCs were then fed into DAPC to return a “yes/no” for the presence of COVID-19. Predictive models differed by the number of PCs fed into DAPC, and these were evaluated for performance by leave-one-out cross-validation. Results are shown in FIG. 3A for a model designed to separate all classes in Table 1 using 99% of the dataset variance (available in the top 20 PCs). This plot was effective in showing cluster separation, particularly that the COVID-19 cluster separated from the Healthy cluster more effectively than the BCa group did. The separation of COVID-19 and the non-COVID-19 groups for this model is shown in FIG. 3B. When cross-validated with leave-one-out, an overall prediction accuracy of 97.6% for this dataset. The sensitivity of this model for detecting COVID-19 was 90.9%. The specificity was 98.8%, the positive predictive value (PPV) was 93.0%, and the negative predictive value (NPV) was 95.8%. Finally, the performance of several DAPC models is given in FIG. 3C and illustrate the influence of model architecture on performance. It is noted that at least 6 PCs were required to obtain overall prediction accuracy, sensitivity, specificity, positive predictive value, and negative predictive value above 50%.

Next, the locations of the ISREA nodes were varied with the objective of reducing the number of PCs required in DAPC. Several iterations were performed, and node positions of 400, 439, 446, 605, 1045, 1163, 1247, 1443, 1739, 1768, and 1775 cm⁻¹ yielded similar results to those reported above; however, only 4 PCs of the dataset were required (as opposed to 20). A comparison of the two node sets for detecting the presence of COVID-19 in urine with Rametrix® analysis is shown in Table 2.

TABLE 2
Detection of COVID-19 in urine by Rametrix ® given
two different ISREA node sets

| ISREA | | Overall | Sensi- | Speci- | | |
|---|---|---|---|---|---|---|
| Nodes (cm⁻¹) | PCs | Accuracy | tivity | ficity | PPV | NPV |
| 400, 950, | 20 | 97.6% | 90.9% | 98.8% | 93.0% | 95.8% |
| 1100, 1500, | | | | | | |
| 1800 | | | | | | |
| 400, 439, 446, | 4 | 97.6% | 93.2% | 98.4% | 91.1% | 98.8% |
| 605, 1045, | | | | | | |
| 1163, 1247, | | | | | | |
| 1443, 1739, | | | | | | |
| 1768, 1775 | | | | | | |

The separation of COVID-19 and non-COVID-19 cluster separation in FIG. 3B was investigated further through PC and canonical (DAPC) loadings. Significant Raman shifts (defined as above 0.2% total contribution) are given in Table 3. The molecular assignments were obtained by a published database (Talari, A. et al. Raman spectroscopy of biological tissues. Appl. Spectrosc. Rev. 2015; 50(1): 46-111). It was observed the occurrences of lipids/cholesterol, collagen, and phosphates occurred more regularly than in similar analyses of CKD and BCa (Senger, PLoS One 2020; Huttanus, 2020).

TABLE 3
Molecular assignments for Raman shifts leading
to cluster separations in FIGS. 3A-B.

| Raman Shift | Present in PCA, | Molecular Assignment |
|---|---|---|
| (cm⁻¹) | DAPC, or Both | (Talari, 2015) |
| 425 | Both | N/A |
| 445 | Both | N-C-S stretch |
| 485 | Both | Glycogen |
| 518 | DAPC | Phosphatidylinositol |
| 614 | DAPC | Cholesterol ester |
| 621 | Both | C-C twisting of phenylalanine |
| 627 | DAPC | N/A |
| 682 | DAPC | N/A |
| 688 | PCA | N/A |
| 702 | DAPC | Cholesterol ester |
| 719 | PCA | Lipids |
| 776 | PCA | Phosphatidyl inositol |
| 782 | PCA | DNA |
| 810 | PCA | Phosphodiester |
| 817 | PCA | Collagen |
| 830 | PCA | Phosphate stretching, Tyrosine |
| 847 | PCA | Monosaccharides |
| 860 | DAPC | Phosphate group |
| 880 | Both | Tryptophan |
| 893 | PCA | C-C backbone |
| 900 | DAPC | N/A |
| 906 | DAPC | Tyrosine |
| 913 | DAPC | Glucose |
| 955 | PCA | Carotenoids |
| 980 | Both | Beta-sheet proteins |
| 992 | DAPC | Red blood cell, |
| | | phenylalanine, NADH |
| 1002 | Both | Urea |
| 1006 | Both | Carotenoids (absent in |
| | | normal tissue) |
| 1008 | DAPC | Phenylalanine |
| 1013 | DAPC | N/A |
| 1030 | DAPC | Phenylalanine of collagen |
| 1049 | DAPC | Glycogen |
| 1058 | PCA | Lipids |
| 1073 | PCA | Fatty acids |
| 1077 | DAPC | Lipids, phospholipids, phosphate |
| 1080 | DAPC | Phospholipids, phosphate, |
| | | collagen, tryptophan |
| 1104 | PCA | Phenylalanine |
| 1107 | PCA | N/A |
| 1126 | Both | Protein, disaccharides, lipids |
| 1185 | PCA | Phosphate |
| 1240 | DAPC | RNA, phosphate, collagen |
| 1327 | Both | Nucleic acids |
| 1396 | Both | Beta-carotene |
| 1491 | PCA | Amino radical cations |
| 1607 | Both | Tyrosine and phenylalanine |
| 1630 | PCA | N/A |
| 1641 | DAPC | N/A |

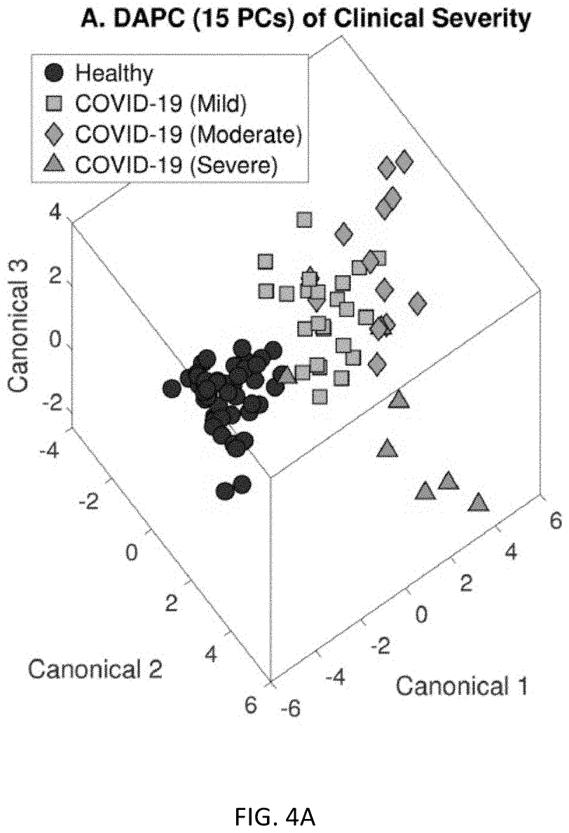

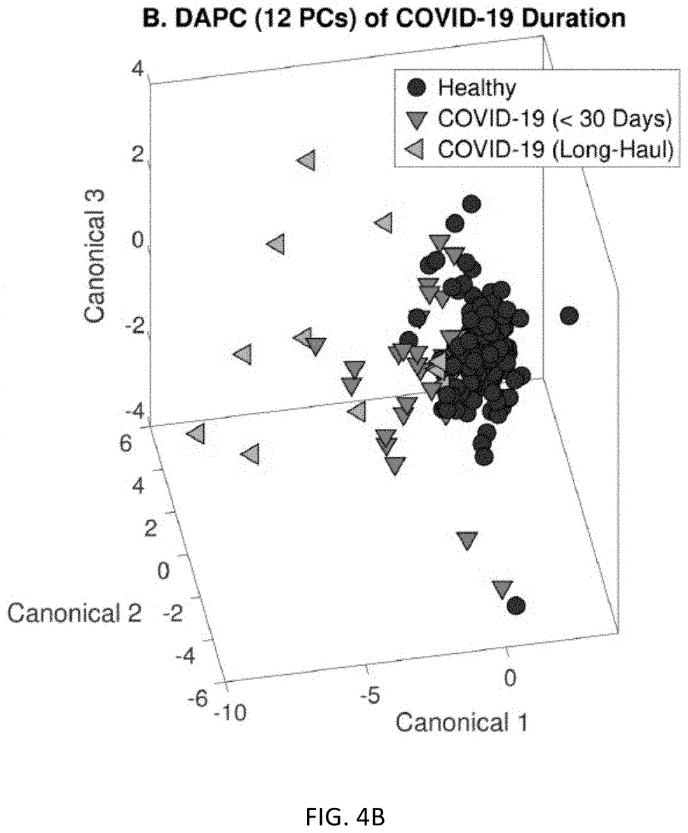

The separation of COVID-19 urine Raman scans by severity of clinical symptoms from those of healthy volunteers is shown in FIG. 4A. When clustered by DAPC, the COVID-19 data largely separated by severity, with mild symptoms clustering closer to healthy scans, on average. However, one urine sample from a COVID-19 patient with severe symptoms clustered with the healthy group. This is unexplained by this analysis. With separation by severity, the presence of COVID-19 was detected with 93.5% overall accuracy (87.5% sensitivity, 100% specificity, 100% PPV, and 88.0% NPV). When inspecting among COVID-19 samples to determine severity, 60-66% overall accuracy was achieved for the three levels (mild, moderate, and severe). Full results are given in Table 4. Given the three levels of severity, the random chance of correct prediction is 33%.

TABLE 4
Prediction of COVID-19 clinical severity
by Rametrix ® analysis of urine.

| Clinical Severity¹ | Accuracy | Sensitivity | Specificity | PPV | NPV |
|---|---|---|---|---|---|
| Mild | 59.1% | 79.2% | 35% | 59.4% | 58.3% |
| Moderate | 65.9% | 61.5% | 67.7% | 44.4% | 80.8% |
| Severe | 65.9% | 57.1% | 67.6% | 25% | 89.3% |

¹Random chance of correct prediction is 33%

Of the patients treated for COVID-19, 26% (12/46) showed symptoms longer than 30 days. Of this group, over 83% showed symptoms for longer than 60 days (up to 300 days). Thus, this group of patients was defined as ‘COVID-19 (Long COVID19)’. A molecular fingerprint was sought to determine if urine metabolomic differences existed between the Long COVID19 group and those whose symptoms resolved in less than 30 days (regardless of clinical severity). Using the initial ISREA node set (400, 950, 1100, 1500, and 1800 cm⁻¹), no significance was found, where prediction accuracy, sensitivity, specificity, PPV, and NPV all exceeded 50% (the random chance of correct prediction). To find a signal, the truncation range was shortened to 600-1800 cm⁻¹ and additional ISREA nodes were searched for. Ultimately, a node set was located that led to 70% sensitivity, with better than 98% specificity, for detecting Long-Haul COVID-19. The results are shown in FIG. 4B, and a comparison of the ISREA node sets with prediction metrics is given in Table 5. The plot in FIG. 4B was constructed with 12 PCs (instead of 4) to better show the separation of Long-Haul samples.

TABLE 5
Detection of long COVID-19 in urine by Rametrix ® given
two different ISREA node sets.

| ISREA | | Overall | Sensi- | Speci- | | |
|---|---|---|---|---|---|---|
| Nodes (cm⁻¹) | PCs | Accuracy¹ | tivity | ficity | PPV | NPV |
| 400, 950, | 12 | 95.1% | 25.0% | 98.7% | 50.0% | 96.2% |
| 1100, 1500, | | | | | | |
| 1800 | | | | | | |
| 600, 1074, | 4 | 97.6% | 70.0% | 98.7% | 70.0% | 98.7% |
| 1153, 1230 | | | | | | |

¹Random chance of correct prediction is 50%.

These results demonstrate that SARS-CoV-2 infection changes the chemical composition of urine. These changes—complex, multimolecular ‘fingerprints’—can be detected using Raman spectroscopic examination and computational analysis. Sample analysis is low-cost (dollars per sample) and rapid (results <30 minutes). This analytical method does not detect virus or viral components. This method also does not identify a single “biomarker” of COVID-19 disease, but rather a “biomarker pattern” composed of molecular clusters associated with disease. These biomarker patterns reflect systemic inflammatory, immunologic, and metabolic reactions to infection. It is hypothesized that viral infection of the kidney (if substantiated) may affect renal form/function and urine composition. These results support information in many of the studies reviewed/critiqued by Hassler, et. al. (Hassler, 2021).

This method of analysis could provide a validated, non-invasive, inexpensive method to monitor systemic manifestations of disease. This could be used to detect infections potentially missed with current PCR/antigen-based technologies and to monitor the efficacy of therapy and/or detect possible disease progression. The method could also prove to be useful tool for monitoring direct/indirect renal effects of COVID-19 disease. This technology could easily be used for non-invasive, repetitive monitoring of individuals who choose not to be vaccinated, to detect ‘break-through’ infections in vaccinated individuals, and to differentiate COVID-19 disease from seasonal respiratory infections (influenza), and may be especially useful in the detection and management of Long COVID19.

Raman spectra from urine specimens from 46 symptomatic COVID-19 patients with positive real time-polymerase chain reaction (RT-PCR) tests for infection or household contact with test-positive patients were compared with urine Raman spectra from healthy individuals (n=185), peritoneal dialysis patients (n=20), and patients with active bladder cancer (n=17), collected between 2016-2018 (i.e., pre-COVID-19). All urine Raman spectra were also compared with urine specimens collected from healthy, fully vaccinated volunteers (n=19) from July to September 2021. Disease severity (primarily respiratory) ranged among mild (n=25), moderate (n=14), and severe (n=7). Seventy percent of patients sought evaluation within 14 days of onset. One severely affected patient was hospitalized, the remainder being managed with home/ambulatory care. Twenty patients had clinical pathology profiling. Seven of 20 patients had mildly elevated serum creatinine values (>0.9 mg/dl; range 0.9-1.34 mg/dl) and 6/7 of these patients also had estimated glomerular filtration rates (eGFR)<90 mL/min/1.73 m² (range 59-84 mL/min/1.73 m²). The present technology (Raman Chemometric Urinalysis—Rametrix®) had an overall prediction accuracy of 97.6% for detecting complex, multimolecular fingerprints in urine associated with COVID-19 disease. The sensitivity of this model for detecting COVID-19 was 90.9%. The specificity was 98.8%, the positive predictive value was 93.0%, and the negative predictive value was 98.4%. In assessing severity, the method showed to be accurate in identifying symptoms as mild, moderate, or severe (random chance=33%) based on the urine multimolecular fingerprint. Finally, a fingerprint of ‘Long COVID-19’ symptoms (defined as lasting longer than 30 days) was located in urine. The methods were able to locate the presence of this fingerprint with 70.0% sensitivity and 98.7% specificity in leave-one-out cross-validation analysis.

Any method or algorithm described herein can be embodied in software or set of computer-executable instructions capable of being run on a computing device or devices. The computing device or devices can include one or more processor (CPU) and a computer memory. The computer memory can be or include a non-transitory computer storage media such as RAM which stores the set of computer-executable (also known herein as computer readable) instructions (software) for instructing the processor(s) to carry out any of the algorithms, methods, or routines described in this disclosure. As used in the context of this disclosure, a non-transitory computer-readable medium (or media) can include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM. The computer-readable instructions can be programmed in any suitable programming language, including JavaScript, C, C#, C++, Java, Python, Perl, Ruby, Swift, Visual Basic, and Objective C. Embodiments of the invention also include a non-transitory computer readable storage medium having any of the computer-executable instructions described herein.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising any combination of software, hardware, or firmware.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method for determining COVID-19 status of a subject, comprising:
obtaining a test Raman spectrum from a urine sample from a test subject;
obtaining at least one positive reference Raman spectrum from a urine sample from a COVID-19 positive subject and at least one negative reference Raman spectrum from a urine sample from a COVID-19 negative subject;
identifying at least one significant Raman shift;
analyzing the test Raman spectrum, positive Raman spectrum, and negative Raman spectrum by performing principal component analysis (PCA) and/or discriminant analysis of principal components (DAPC) using the at least one significant Raman shift; and
classifying the test subject as COVID-19 positive or COVID-19 negative based on the analyzing.

2. The method of claim 1, further comprising processing the test Raman spectrum, positive Raman spectrum, and/or negative Raman spectrum to obtain a processed test Raman spectrum, a processed positive Raman spectrum, and/or a processed negative Raman spectrum.

3. The method of claim 2, wherein the processing comprises a baseline correction.

4. The method of claim 3, wherein the baseline correction is performed using ISREA.

5. The method of claim 4, wherein the baseline correction comprises selecting one or more ISREA nodes selected from 400, 439, 446, 605, 950, 1045, 1100, 1163, 1247, 1443, 1500, 1739, 1768, 1775, and 1800 cm⁻¹.

6. The method of claim 2, wherein the processing comprises truncating the test Raman spectrum, positive Raman spectrum, and/or negative Raman spectrum.

7. The method of claim 6, wherein the truncating is performed between about 600-1,800 cm⁻¹.

8. The method of claim 1, wherein the significant Raman shift is defined as a shift having above 0.2% of a total contribution to a Raman spectrum.

9. The method of claim 1, wherein the at least one significant Raman shift is selected from 425 cm⁻¹, 445 cm⁻¹, 485 cm⁻¹, 518 cm⁻¹, 614 cm⁻¹, 621 cm⁻¹, 627 cm⁻¹, 682 cm⁻¹, 688 cm⁻¹, 702 cm⁻¹, 719 cm⁻¹, 776 cm⁻¹, 782 cm⁻¹, 810 cm⁻¹, 817 cm⁻¹, 830 cm⁻¹, 847 cm⁻¹, 860 cm⁻¹, 880 cm⁻¹, 893 cm⁻¹, 900 cm⁻¹, 906 cm⁻¹, 913 cm⁻¹, 955 cm⁻¹, 980 cm⁻¹, 992 cm⁻¹, 1002 cm⁻¹, 1006 cm⁻¹, 1008 cm⁻¹, 1013 cm⁻¹, 1030 cm⁻¹, 1049 cm⁻¹, 1058 cm⁻¹, 1073 cm⁻¹, 1077 cm⁻¹, 1080 cm⁻¹, 1104 cm⁻¹, 1107 cm⁻¹, 1126 cm⁻¹, 1185 cm⁻¹, 1240 cm⁻¹, 1327 cm⁻¹, 1396 cm⁻¹, 1491 cm⁻¹, 1607 cm⁻¹, 1630 cm⁻¹, and 1641 cm⁻¹.

10. The method of claim 1, wherein the analyzing further comprises identifying statistically significant differences between the test Raman spectrum and the positive and/or negative reference spectra.

11. The method of claim 10, wherein the identifying statistically significant differences comprises performing one or more of total canonical distance (TCD), total principal component distance (TPD), or total spectra distance (TSD).

12. A method of identifying a condition of a subject, comprising:
obtaining Raman spectra from a urine sample from a subject;
comparing the Raman spectra of the urine sample to a selected model;
wherein the selected model is constructed from various Raman spectra of urine from individuals having and not having COVID-19, and by:
(a) applying baseline correction to a range of wavenumbers of the various Raman spectra to obtain baseline corrected Raman spectra;
(b) performing normalization of the baseline corrected Raman spectra to obtain normalized Raman spectra;
(c) performing principal component analysis (PCA) of the normalized Raman spectra to identify principal components (PCs) of the urine from the individuals having and not having COVID-19;
(d) performing one or more analysis selected from discriminant analysis of principal components (DAPC), Partial Least Squares (PLS), machine learning, and/or neural networks (NN), to obtain one or more chemometric models based on one or more of the PCs and for the DAPC analysis, comprising canonicals equal in number to the PCs;
(e) for the DAPC analysis, determining a fractional contribution of each wavenumber to each canonical of one or more of the chemometric models to determine which wavenumbers give rise to separations seen in a plot of two or more of the canonicals; and
(f) identifying statistically significant spectral differences between the urine from the individuals having the specified condition and the urine from individuals not having COVID-19 by performing total principal component distance (TPD) and/or total spectral distance (TSD);
wherein the comparing of the Raman spectra of the urine sample to the selected model comprises identifying whether the urine sample is classified according to the selected model as being urine either from a subject who has or does not have COVID-19.

13. The method of claim 12, wherein the baseline correction is performed using ISREA.

14. The method of claim 13, wherein the baseline correction comprises choosing ISREA nodes, wherein the ISREA nodes are selected from 400, 950, 110, 1500, and 1800 cm⁻¹.

15. The method of claim 12, wherein multiple chemometric models are obtained with different numbers of principal components.

16. The method of claim 15, further comprising testing one or more of the chemometric models using a leave-one-out cross-validation technique to select one of the chemometric models as the selected model.

17. The method of claim 12, wherein the selected model is one where the DAPC model is based on up to about 20 PCs.

18. The method of claim 12, wherein the selected model is one where the DAPC model is based on up to about 5 PCs.

19. The method of claim 12, wherein the selected model is constructed from various Raman spectra from individuals classified as having mild COVID-19 symptoms lasting less than 30 days, moderate COVID-19 symptoms lasting less than 30 days, severe COVID-19 symptoms lasting less than 30 days, or COVID-19 symptoms lasting 30 days or more.

20. The method of claim 19, wherein when the urine sample is classified according to the selected model as being urine from a subject who has COVID-19, further classifying the urine sample as being from a subject having mild COVID-19 symptoms lasting less than 30 days, moderate COVID-19 symptoms lasting less than 30 days, severe COVID-19 symptoms lasting less than 30 days, or COVID-19 symptoms lasting 30 days or more.
